(19) United States
(12) Patent Application Publication
(10) Pub. No.: US 2019/0269604 A1
(43) Pub. Date: Sep. 5, 2019

(54) COSMETIC COMPOSITION WITH SELF-FOAMING FUNCTION

(71) Applicant: COSMAX, INC., Gyeonggi-do (KR)

(72) Inventors: Chan Gu KIM, Gyeonggi-do (KR); Ja Yoon KOO, Gyeonggi-Do (KR); Ho Yeol KIM, Gyeonggi-Do (KR); Yoon Nam JO, Gyeonggi-do (KR); Il Rang LEE, Gyeonggi-Do (KR); Chun Jin PARK, Gyeonggi-do (KR); Youn Joon KIM, Seoul (KR)

(21) Appl. No.: 16/291,280

(22) Filed: Mar. 4, 2019

(30) Foreign Application Priority Data
Mar. 5, 2018 (KR) 10-2018-0025846

(51) Int. Cl. A61K 8/891; A61K 8/04; A61K 8/58; A61K 8/31; A61K 8/34; A61K 8/44; A61K 8/73; A61K 8/81; A61Q 19/10; A61Q 19/00; A61K 8/02

(52) U.S. Cl. CPC A61K 8/891; A61K 8/046; A61K 8/585; A61K 8/31; A61K 8/345; A61K 2800/30; A61K 8/73; A61K 8/8158; A61Q 19/10; A61Q 19/007; A61K 8/0212; A61K 8/44

(57) ABSTRACT

The present invention relates to a cosmetic composition containing volatile oil for skin care. The cosmetic composition contains volatile oil instead of a fluorine compound, and thus provides a cleansing effect, such as removing sebum and waste substances, and a skin care effect, such as sebum improvement or skin texture improvement, through bubbles by a self-foaming function. Furthermore, the cosmetic composition does not a fluorine compound, which is difficult to manage as a raw material for cosmetic products in the prior art, and thus is expected to further reduce environmental harmfulness.

### CROSS-REFERENCE TO RELATED APPLICATION

This application claims priority to and the benefit of Korean Patent Application No. 10-2018-0025846, filed on Mar. 5, 2018, the disclosure of which is incorporated herein by reference in its entirety.

### FIELD

The present disclosure relates to a cosmetic composition with a self-foaming function and, specifically, to a cosmetic composition containing volatile oil without containing a fluorine compound.

### BACKGROUND

Skin plays a very important role as a barrier function to protect an individual from the outside. The barrier function is a protective function of protecting against various stimuli (chemicals, air pollutants, arid environments, ultraviolet light, and the like) from the outside and preventing excessive evaporation of body moisture through skin, and such a protective function can be maintained only when the stratum corneum composed of keratinocytes is normally formed.

The stratum corneum (horny layer), which is the outermost part of the epidermis, is formed of keratinocytes, and is composed of completely differentiated keratinocytes and lipid layers surrounding the keratinocytes. The keratinocytes are characteristic cells in which basal cells proliferating continuously in the lowest epidermis gradationally undergo morphological and functional changes and rise to the surface of the skin. After a predetermined period of time, old keratinocytes are separated from the skin and new keratinocytes replace functions of the old keratinocytes, and a series of these repetitive changes is called “differentiation of epidermal cells” or “keratinization”. During the keratinization procedure, keratinocytes form the stratum corneum while producing natural moisturizing factors (NMFs) and intercellular lipids (ceramides, cholesterols, and fatty acids), so that the stratum corneum is firm and flexible, and thus retains functions as a skin barrier.

The stratum corneum may easily lose functions thereof due to lifestyle factors, such as excessive face washing or bathing, environmental factors, such as dry air pollutants, and endogenous diseases, such as atopic skin or elderly skin. In fact, due to the increasing number of various factors in modern times, the appeals of dry skin symptoms and general disabilities resulting therefrom are recently on the rise.

Meanwhile, a self-foamable composition forms bubbles when applied, and thus can be used for face washing or the like. Such a formulation type composition can have foaming power upon application since a self-foaming agent is generally dissolved in a formation. The greatest feature of such a self-foamable composition is in the visually formed bubbles, which give a massage effect due to the nature of the formulation, and thus is positively perceived by consumers. In addition, this self-foamable composition is recently used for cleansing products as well as skin care products, such as creams, packs, and essences.

In conventional techniques associated with self-foamable compositions, fluorine compounds are used in self-foaming for convenient and esthetic effects. The use of such fluorine compounds is gradually diminished since the fluorine compounds managed to be of cosmetic grades are rare and are likely to be environmental pollution sources.

### SUMMARY

### Technical Problem

The present inventors confirmed that a cosmetic composition containing volatile oil can have a skin care effect by a self-foaming function even without using a fluorine compound, and thus completed the present invention.

Therefore, an aspect of the present invention is to provide a cosmetic composition for skin care, the cosmetic composition containing volatile oil as a self-foaming agent.

Another aspect of the present invention is to provide a use of a cosmetic composition for skin care, the cosmetic composition containing volatile oil as a self-foaming agent.

Still another aspect of the present invention is to provide a skin care method using a cosmetic composition containing volatile oil as a self-foaming agent.

### Technical Solution

The present invention has been made in view of the above-mentioned problems, such as environmental harmfulness, of an existing self-foamable cosmetic composition employing a fluorine compound, and the present inventors have developed a cosmetic composition having a low possibility of environmental contamination while retaining a self-foaming function, by using volatile oil, which is managed to be of cosmetic grade, instead of the fluorine compound.

In accordance with an aspect of the present invention, there is provided a cosmetic composition for skin care, the cosmetic composition containing volatile oil as a self-foamable agent.

The cosmetic composition may not contain a fluorine compound.

The cosmetic composition may contain 0.1-20 wt % of volatile oil. When the volatile oil is contained in the above range, the cosmetic composition has a self-foaming effect through a surfactant contained in a formulation.

The volatile oil may be disiloxane, trisiloxane, dimethicone, and/or isopentane, but is not limited thereto.

The foregoing kinds of volatile oil may be used alone or in a combination thereof, or all of the foregoing kinds of volatile oil may be used.

When two kinds of the volatile oil are contained, the two kinds of the volatile oil may be mixed at a weight ratio of 1:1-5, for example, 1:1.5.

When three kinds of the volatile oil are contained, the three kinds of the volatile oil may be mixed at a weight ratio of 1:1:1-5 or 1:1-5:1, for example, 1:1:1.5 or 1:1.5:1.

The cosmetic composition may contain 0.01-20 wt % of a surfactant. The surfactant, when being contained in the above range, meets the volatile oil to show a self-foaming effect.

The surfactant may be sodium cocoyl apple amino acid, potassium cocoyl glycinate, decyl glucoside, sodium lauryl glucose carboxylate, sodium cocoyl glutamate, cocamidopropyl betaine, sodium cocoamphoacetate, and/or lauryl glucoside, and may be, for example, sodium cocoyl apple amino acid, and/or potassium cocoyl glycinate, but is not limited thereto.

The cosmetic composition may contain 0.01-20 wt % of a moisturizer. The surfactant, when being contained in the above range, produces a moisture retaining effect, to prevent a user from feeling stretched even after face washing.

The moisturizer may be glycerin, 1,2-hexanediol, ethylhexyl glycerin, 1,3-butylene glycol, dipropylene glycol, isopentyl glycol, propanediol, betaine, and/or sorbitol, and may be, for example, glycerin, 1,2-hexanediol, and/or ethylhexyl glycerin, but is not limited thereto.

The cosmetic composition may contain 0.01-10 wt % of a gellant. The gellant, when being contained in the above range, produces an effect of maintaining stability of a formulation.

The gellant may be at least one selected from the group consisting of xanthan gum, acrylate copolymer, acrylate/C10-30 alkyl acrylate crosspolymer, sodium polyacrylate, carbomer, and cellulose gum, and may be, for example, xanthan gum, but is not limited thereto.

The cosmetic composition may contain 10-95 wt % of purified water. The “purified water” refers to water obtained by distillation or ion exchange, and means pure water that is used for the purpose of providing water not containing any agent or other minerals.

In a specific embodiment, the cosmetic composition may contain 0.1-20 wt % of oil, 0.01-20 wt % of a surfactant, 0.01-20 wt % of a moisturizer, 0.01-10 wt % of a gellant, and 10-95 wt % of water, but is not limited thereto.

The cosmetic composition according to the present invention may contain the ingredients that are ordinarily used in a cosmetic composition, and may contain, for example, ordinary adjuvants, such as a metal ion sequestering agent, a preservative, a thickener, and a fragrance, and a carrier.

In addition, the cosmetic composition according to the present invention may be prepared in the form of a general formulation in the art, for example, an emulsified formulation or a solubilized formulation. Examples of the emulsified formulation may be a nourishing lotion, a cream, an essence, and the like, and examples of the solubilizer formulation may be a softening lotion and the like. In addition, the cosmetic composition of the present invention may be prepared in the form of an adjurvant that can be locally or systemically applied and is ordinarily used in a field of dermatology, as well as a cosmetic product, by containing a dermatologically acceptable medium or base.

Examples of an appropriate formulation for a cosmetic product may be a solution, a gel, a solid or kneaded anhydrous product, an emulsion obtained by dispersing oil in water, a suspension, a microemulsion, a microcapsule, microgranules, an ionic (liposome) or non-ionic vesicle dispersant, a cream, a toner, a lotion, a powder, an ointment, a spray, or a concealer stick. The cosmetic composition may be prepared in the form of a foam, or in the form of an aerosol composition further containing a compressed propellant.

Examples of a product to which the cosmetic composition of the present invention can be added include a skin lotion, a skin softener, a skin toner, an astringent, a lotion, a milk lotion, a moisture lotion, a nourishing lotion, a massage cream, a nourishing cream, a moisture cream, a hand cream, an essence, a nourishing essence, a pack, a soap, a shampoo, a cleansing foam, a cleansing lotion, a cleansing cream, a body lotion, a body cleanser, a serum, a pressed powder, a loose powder, a patch, a sprayer, and the like.

The cosmetic composition may be impregnated into a mask sheet. When the cosmetic composition may be impregnated into a mask sheet, a material of the mask sheet may be a material including at least one selected from the group consisting of cotton, a nonwoven fabric, rayon, and cellulose, and may be, for example, a material composed of a nonwoven fabric, but is not limited thereto.

In accordance with another aspect of the present invention, there is provided a method for preparing a cosmetic composition for skin care, the cosmetic composition containing volatile oil as a self-foaming agent.

A water phase containing a moisturizer and purified water is completely dissolved, and then a gellant is added, followed by stirring.

The mixture is cooled to 25° C. or lower after the stirring. The reason is that the preparation of the contents is performed in a condition lower than the volatilization temperature of a volatile substance.

Volatile oil is introduced to the contents while stirring is carried out using a homo mixer, thereby preparing an emulsion.

Stirring is carried out while a surfactant is introduced into the emulsion, thereby obtaining a gel-phase or liquid-phase composition.

Additionally, as needed, the composition and a mask sheet are placed in a pouch, followed by sealing and filing, thereby preparing a mask sheet type cosmetic composition for skin care.

In accordance with still another aspect of the present invention, there is provided a use of a cosmetic composition for skin care, the composition containing volatile oil as a self-foaming agent.

In accordance with still another aspect of the present invention, there is provided a skin care method using a cosmetic composition containing volatile oil as a self-foaming agent.

The method may contain contacting the cosmetic composition containing volatile oil as a self-foaming agent to a subject.

The subject may be, but is not limited to, mammals including primates including humans, monkeys, and rodents including mice, rats, and the like.

The contact refers to a direct contact of the cosmetic composition containing volatile oil as a self-foaming agent to or near the body part of the subject, for example, a cosmetic composition containing a volatile oil as a self-foaming agent is applied to human skin.

The overlapping descriptions of the cosmetic composition, the use for skin care, and the skin care method are omitted in consideration of the complexity of the present specification.

### Advantageous Effects

The present invention is directed to a cosmetic composition containing volatile oil for skin care. The cosmetic composition contains volatile oil instead of a fluorine compound, and thus provides a cleansing effect, such as removing sebum and waste substances, and a skin care effect, such as sebum improvement or skin texture improvement, through bubbles by a self-foaming function. The cosmetic composition does not a fluorine compound, which is difficult to manage as a raw material for cosmetic products in the prior art, and thus is expected to further reduce environmental harmfulness.

### BRIEF DESCRIPTION OF THE DRAWINGS

FIG. 1 shows a sebum improvement effect of a cosmetic composition of the present invention;

FIG. 2 shows a pore cleansing effect of a cosmetic composition of the present invention;

FIG. 3A and FIG. 3B shows a skin texture improvement effect of a cosmetic composition of the present invention; and

FIG. 4 shows a stratum corneum removal effect of a cosmetic composition of the present invention.

### DETAILED DESCRIPTION

Hereinafter, the present invention will be described in detail with reference to examples. These examples are only for illustrating the present invention more specifically, and it will be apparent to those skilled in the art that the scope of the present invention is not limited by these examples.

Throughout the present specification, the term “%” used to express the concentration of a specific material, unless otherwise particularly stated, refers to (wt/wt) % for solid/solid, (wt/vol) % for solid/liquid, and (vol/vol) % for liquid/liquid.

### Preparative Example 1: Cosmetic Composition not Containing Fluorine Compound

A cosmetic composition not containing a fluorine compound was prepared by the following method. A specific composition is shown in Table 1 below.

TABLE 1

| | | | Content |
|---|---|---|---|
| No. | Use | Ingredient | (wt %) |
| 1 | Metal ion sequestering | Purified water | to 100¹⁾ |
| | agent | | |
| 2 | Metal ion sequestering | Disodium EDTA | 0.02 |
| | agent | | |
| 3 | Moisturizer | Glycerin | 5 |
| 4 | Gellant | Xanthan gum | 0.2 |
| 5 | Moisturizer | 1,2-Hexandiol/ | 2.0/0.04 |
| | | Ethylhexyl glycerin | |
| 6 | Emulsifier/Gellant | Polyacrylamide | 1 |
| 7 | Fragrance | Fragrance | 0.05 |
| 8 | Volatile oil | At least one of disiloxane, | 5 |
| | | trisiloxane, and isopentane | |
| 9 | Surfactant | Sodium cocoyl apple amino | 3 |
| | | acid | |
| 10 | Surfactant | Potassium cocoyl glycinate | 5 |

¹⁾Part of the entire content (100) excluding the other raw materials being filled with purified water.

Disodium EDTA, glycerin, xanthan gum, and a preservative were added to purified water, and dissolved with stirring. After confirmation of complete dissolution, polyacrylamide was added with stirring using a homo mixer, and then, stirring was carried out until the polyacrylamide was completely dissolved. After confirmation of dissolution without agglomeration, a fragrance was added and stirred. After stirring for 3 minutes, the mixture was cooled to 25° C. Volatile oil (disiloxane, trisiloxane, dimethicone and/or isopentane) was added with stirring, and thus emulsified. After confirmation of the emulsification, sodium cocoyl apple amino acid and potassium cocoyl glycinate were added with stirring using only paddles without using a homo mixer.

1-1. Use of Volatile Oil Alone

A cosmetic composition was prepared by the method as in Preparative Example 1 above while disiloxane was used as volatile oil.

1-2. Use of Volatile Oils in Combination

A cosmetic composition was prepared by the method as in Preparative Example 1 above while disiloxane and trisiloxane (3.0 and 2.0 wt %, respectively) were used as volatile oil.

### Comparative Example 0: Preparation of Cosmetic Composition not Containing Fluorine Compound

A cosmetic composition was prepared by the method as in Preparative Example 1 above while isopentane was used as volatile oil. A specific composition is shown in Table 1 above.

### Comparative Example 1: Cosmetic Composition Containing Fluorine Compound

A cosmetic composition containing a fluorine compound was prepared by the following method. A specific composition is shown in Table 2 below.

TABLE 2

| | | | Content |
|---|---|---|---|
| No. | Use | Ingredient | (wt %) |
| 1 | Metal ion sequestering | Purified water | to 100 |
| | agent | | |
| 2 | Metal ion sequestering | Disodium EDTA | 0.02 |
| | agent | | |
| 3 | Moisturizer | Glycerin | 5 |
| 4 | Gellant | Acrylate copolymer | 2 |
| 5 | Moisturizer | 1,2-Hexandiol/ | 2.0/0.04 |
| | | Ethylhexyl glycerin | |
| 6 | Emulsifier/Gellant | Polyacrylamide | 1 |
| 7 | Fragrance | Fragrance | 0.05 |
| 8 | Fluorine compound | Ethyl perfluorobutyl ether, | 5 |
| | | Ethyl perfluoroisobutyl ether | |
| 9 | Surfactant | Sodium cocoyl apple amino | 3 |
| | | acid | |
| 10 | Surfactant | Potassium cocoyl glycinate | 5 |
| 11 | Neutralizing agent | Triethanol amine | 0.2 |

¹⁾ Part of the entire content (100) excluding the other raw materials being filled with purified water

Disodium EDTA, glycerin, an acrylate copolymer, and a preservative were added to purified water, and dissolved with stirring. After confirmation of complete dissolution, polyacrylamide was added with stirring using a homo mixer, and then, stirring was carried out until the polyacrylamide was completely dissolved. After confirmation of dissolution without agglomeration, a fragrance was added and stirred. After the stirring, ethyl perfluorobutyl ether and ethyl perfluoroisobutyl ether were added with stirring, and thus emulsified. After confirmation of the emulsification, sodium cocoyl apple amino acid and potassium cocoyl glycinate were added with stirring using only paddles without using a homo mixer. Thereafter, the separately dissolved acrylate copolymer was added, followed by stirring for 3 minutes, and then triethanol amine was added, followed by further stirring for 3 minutes.

### Example 1: Sensory Tests of Cleansing, Moisturizing, and Softening of Cosmetic Composition

Mask sheet type cosmetic compositions were prepared by depositing the contents, prepared by the methods in Preparative Example 1, Comparative Example 0, and Comparative Example 1, on nonwoven fabrics, and then tested on 20 healthy male/female subjects.

After the manufactured nonwoven fabrics were used for 15 minutes, respectively, sebum improvement, moisturizing feeling, skin softening, and cleaning power were examined, and the results are shown in Table 3 (Preparative Example 1-1), Table 4 (Preparative Example 1-2), Table 5 (Comparative Example 0), and Table 6 (Comparative Example 1). The nonwoven fabrics used in the tests employed a cupra material. Scores were evaluated on the basis of the following criteria.

5 Points: Very good

4 Points: Good

3 Points: Medium

2 Points: Poor

1 Point: Very poor

TABLE 3

| Male | A | B | C | D | E | F | G | H | I | J |
|---|---|---|---|---|---|---|---|---|---|---|
| Sebum improvement | 4 | 5 | 4 | 5 | 5 | 4 | 3 | 4 | 4 | 4 |
| Moisturization | 5 | 5 | 4 | 4 | 3 | 4 | 5 | 4 | 5 | 4 |
| Softening | 4 | 4 | 3 | 4 | 3 | 3 | 4 | 4 | 5 | 3 |
| Cleansing power | 5 | 4 | 4 | 5 | 4 | 5 | 4 | 4 | 5 | 4 |
| Female | K | L | M | N | O | P | Q | R | S | T |
| Sebum improvement | 4 | 4 | 5 | 5 | 4 | 5 | 3 | 4 | 4 | 5 |
| Moisturization | 3 | 4 | 4 | 4 | 5 | 5 | 5 | 4 | 3 | 5 |
| Softening | 3 | 3 | 3 | 4 | 3 | 3 | 4 | 3 | 2 | 5 |
| Cleansing power | 5 | 5 | 5 | 4 | 5 | 4 | 5 | 4 | 5 | 4 |

TABLE 4

| Male | A | B | C | D | E | F | G | H | I | J |
|---|---|---|---|---|---|---|---|---|---|---|
| Sebum improvement | 3 | 4 | 5 | 5 | 5 | 4 | 5 | 4 | 4 | 4 |
| Moisturization | 4 | 3 | 5 | 4 | 4 | 4 | 4 | 4 | 5 | 4 |
| Softening | 4 | 4 | 4 | 4 | 3 | 3 | 4 | 4 | 5 | 3 |
| Cleansing power | 5 | 4 | 4 | 5 | 5 | 3 | 4 | 4 | 5 | 4 |
| Female | K | L | M | N | O | P | Q | R | S | T |
| Sebum improvement | 4 | 4 | 4 | 4 | 4 | 5 | 3 | 4 | 4 | 5 |
| Moisturization | 4 | 4 | 4 | 4 | 4 | 5 | 5 | 4 | 5 | 5 |
| Softening | 4 | 3 | 4 | 3 | 3 | 3 | 4 | 2 | 2 | 5 |
| Cleansing power | 4 | 5 | 5 | 4 | 5 | 4 | 5 | 5 | 3 | 4 |

TABLE 5

| Male | A | B | C | D | E | F | G | H | I | J |
|---|---|---|---|---|---|---|---|---|---|---|
| Sebum improvement | 4 | 4 | 4 | 5 | 5 | 4 | 3 | 4 | 4 | 4 |
| Moisturization | 4 | 5 | 4 | 5 | 4 | 4 | 3 | 3 | 5 | 4 |
| Softening | 2 | 3 | 3 | 3 | 3 | 2 | 2 | 3 | 3 | 2 |
| Cleansing power | 5 | 4 | 5 | 5 | 4 | 4 | 4 | 5 | 4 | 3 |
| Female | K | L | M | N | O | P | Q | R | S | T |
| Sebum improvement | 3 | 5 | 5 | 4 | 5 | 4 | 5 | 4 | 5 | 4 |
| Moisturization | 4 | 5 | 5 | 5 | 4 | 4 | 5 | 4 | 3 | 5 |
| Softening | 3 | 2 | 2 | 3 | 3 | 4 | 3 | 3 | 3 | 3 |
| Cleansing power | 5 | 5 | 5 | 4 | 4 | 5 | 4 | 3 | 4 | 4 |

TABLE 6

| Male | A | B | C | D | E | F | G | H | I | J |
|---|---|---|---|---|---|---|---|---|---|---|
| Sebum improvement | 4 | 4 | 4 | 5 | 5 | 4 | 3 | 4 | 4 | 4 |
| Moisturization | 4 | 5 | 4 | 5 | 5 | 4 | 4 | 3 | 4 | 4 |
| Softening | 3 | 2 | 3 | 3 | 3 | 2 | 2 | 3 | 4 | 2 |
| Cleansing power | 5 | 4 | 5 | 5 | 4 | 4 | 4 | 5 | 4 | 3 |
| Female | K | L | M | N | O | P | Q | R | S | T |
| Sebum improvement | 4 | 5 | 5 | 4 | 5 | 5 | 4 | 4 | 4 | 4 |
| Moisturization | 5 | 5 | 5 | 3 | 4 | 4 | 5 | 4 | 4 | 4 |
| Softening | 3 | 2 | 2 | 3 | 3 | 4 | 3 | 3 | 3 | 3 |
| Cleansing power | 5 | 5 | 5 | 4 | 4 | 5 | 4 | 3 | 4 | 4 |

The comprehensive comparison results of the sensory tests are shown in Table 7 below.

TABLE 7

| | Preparative | Preparative | Comparative | Comparative |
|---|---|---|---|---|
| | Example 1-1 | Example 1-2 | Example 0 | Example 1 |
| Male | | | | |
| Sebum | 4.2 | 4.3 | 4.1 | 4.1 |
| improvement | | | | |
| Moisturization | 4.3 | 4.1 | 4.1 | 4.2 |
| Softening | 3.7 | 3.8 | 2.6 | 2.7 |
| Cleansing | 4.4 | 4.3 | 4.3 | 4.3 |
| power | | | | |
| Female | | | | |
| Sebum | 4.3 | 4.1 | 4.4 | 4.4 |
| improvement | | | | |
| Moisturization | 4.3 | 4.4 | 4.4 | 4.3 |
| Softening | 3.7 | 3.3 | 2.9 | 2.9 |
| Cleansing | 4.6 | 4.4 | 4.3 | 4.3 |
| power | | | | |

As a result, Preparative Example 1-1 and Preparative Example 1-2, not containing a fluorine compound, which is considered to be most important in a self-foaming mask sheet, showed the same performance as Comparative Example 1 containing a fluorine compound, and thus it was observed that all of the male/female subjects felt similar senses of use in view of all of sebum improvement, moisturizing ability, softening, and washing power.

### Example 2: Tests of Sebum Improving, Pore Cleansing, Skin Texture Improving, and Stratum Corneum Removing Effects of Cosmetic Composition

On the basis of the results of Example 1, mask sheet type cosmetic compositions were prepared by depositing the contents, prepared by the method in Preparative Example 1-1, on nonwoven fabrics, and then tested on additional five healthy male/female subjects to examine sebum improving, pore cleansing, skin texture improving, and stratum corneum removing effects.

2-1. Sebum Improvement Test

First, a test site (face) was washed with lukewarm water, and then sebum secretion was induced through routine activities for 1 hour. After waiting for 10 minutes in a constant temperature and humidity room (22±2° C., 40-60% RH), the amount of sebum (before) was measured, and after the use of the product, the amount of sebum (after) was measured by the same method. A change in amount of sebum before/after the use of the product was calculated, and then the efficacy of the product was evaluated.

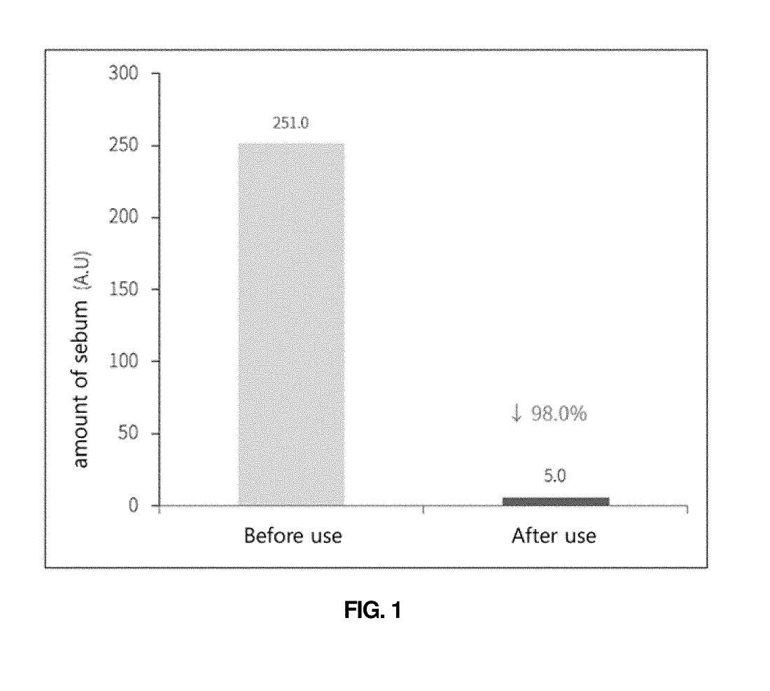

As shown in FIG. 1, the results confirmed that when the composition of the present invention (Preparative Example 1-1) was used, the amount of sebum was reduced by about 98.0% compared with before the use of the composition.

2-2. Pore Cleansing Test

First, a face was washed with lukewarm water, followed by waiting for 10 minutes in a constant temperature and humidity room (22±2° C., 40-60% RH). Residues were allowed to enter pores by using eye shadow on the test site, and then eye shadow residues remaining on a surface of the test site were removed by tape. The pores before and after the test were photographed. The residues were visually checked by using images of the pores before and after the use of the product, and then the efficacy of the product was evaluated.

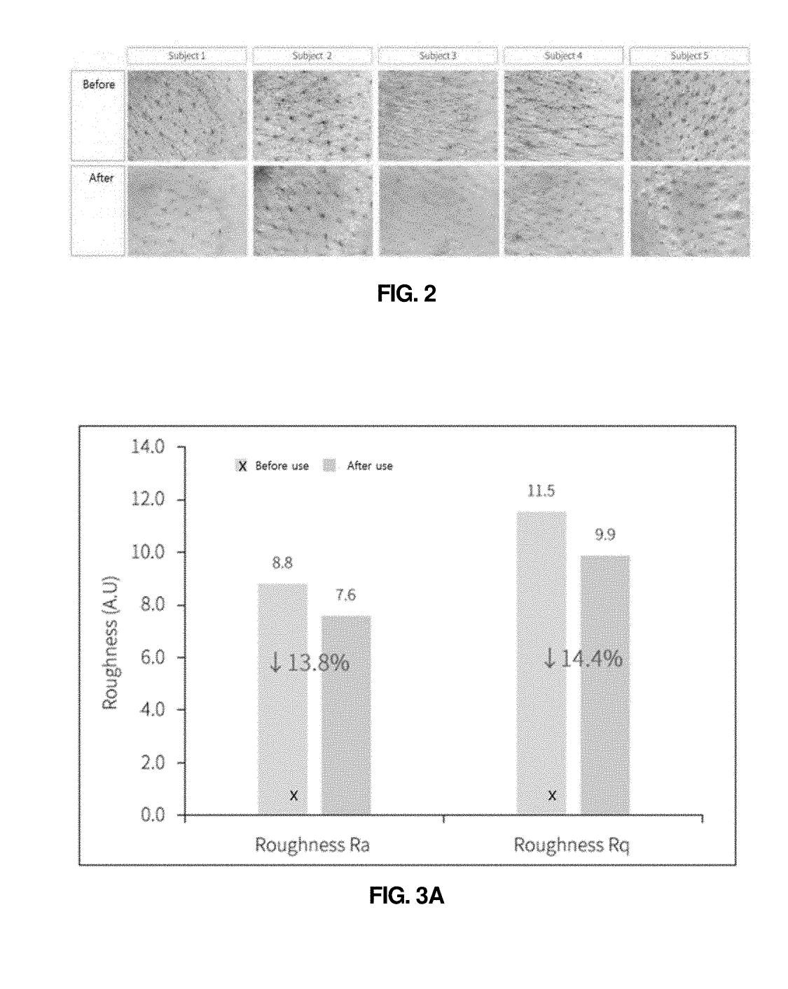

As shown in FIG. 2, the results confirmed that when the composition of the present invention (Preparative Example 1-1) was used, the residues in the pores were reduced.

2-3. Skin Texture Improvement Test

First, a test site (face) was washed with lukewarm water, followed by waiting for 10 minutes in a constant temperature and humidity room (22±2° C., 40-60% RH). Thereafter, the skin texture (before) was photographed, and after the use of the product, the skin texture (after) was measured by the same method. The same area on the front surface of the measurement site was designated, and roughness Ra (roughness scale i: absolute value arithmetic mean of the skin height obtained by making the cross-section of the skin into a waveform and digitizing the waveform) and roughness Rq (roughness scale ii: standard deviation of the skin height obtained by making the cross-section of the skin into a waveform and digitizing the waveform) before and after the use of the product were calculated, and then the efficacy of the product was evaluated.

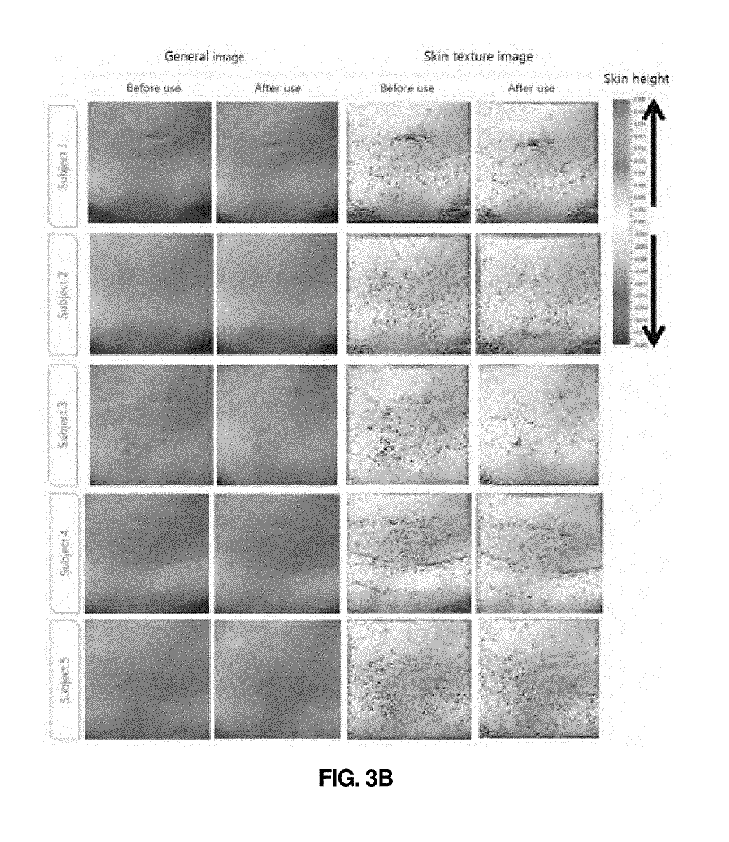

As shown in FIGS. 3a and 3b, the results confirmed that when the composition of the present invention (Preparative Example 1-1) was used, Ra and Rq values were reduced by about 13.8% and about 14.4%, respectively, compared with before the use of the composition.

2-4. Stratum Corneum Removal Test

First, a test site (arm) of the subjects was washed with lukewarm water, followed by waiting for 10 minutes in a constant temperature and humidity room (22±2° C., 40-60% RH). The stratum corneum (before) was collected using B-SQUAM, and after the use of the product, the stratum corneum (after) was collected by the same method. The stratum corneum areas before and after the use of the product were analyzed, and then the efficacy of the product was evaluated.

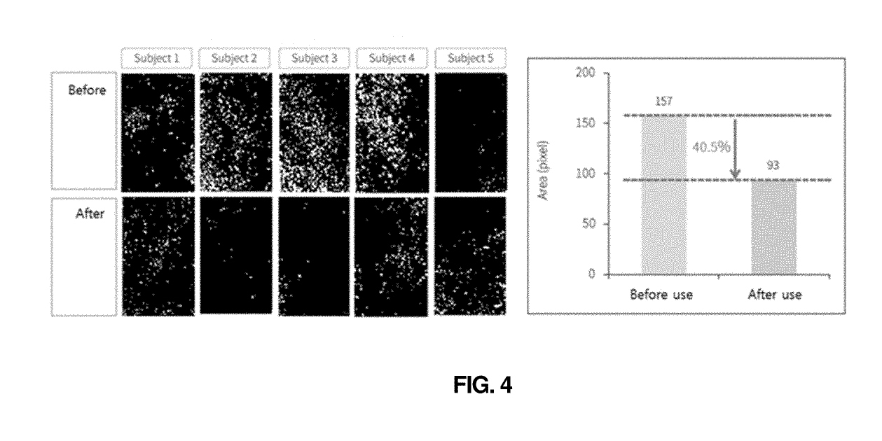

As shown in FIG. 4, the results confirmed that when the composition of the present invention (Preparative Example 1-1) was used, the stratum corneum area was reduced by about 40.5% compared with before the use of the composition.

It can be seen from these results that the present invention employs volatile oil instead of a fluorine compound, and thus the present invention is excellent in view of sebum improvement, moisturizing feeling, skin softening, and cleansing power, and is safe for skin, so a cosmetic composition for skin care can be provided.

What is claimed is:

1. A method for skin care comprising:
contacting a cosmetic composition comprising volatile oil as a self-foaming agent to a subject.

2. The method of claim 1, wherein the cosmetic composition contains no fluorine compound.

3. The method of claim 1, wherein the cosmetic composition contains:
0.1-20 wt % of volatile oil;
0.01-20 wt % of a surfactant;
0.01-20 wt % of a moisturizer;
0.01-10 wt % of a gellant; and
10-95 wt % of purified water.

4. The method of claim 3, wherein the volatile oil is at least one selected from the group consisting of disiloxane, trisiloxane, dimethicone, and isopentane.

5. The method of claim 3, wherein the surfactant is at least one selected from the group consisting of sodium cocoyl apple amino acid, potassium cocoyl glycinate, decyl glucoside, sodium lauryl glucose carboxylate, sodium cocoyl glutamate, cocamidopropyl betaine, sodium cocoamphoacetate, and lauryl glucoside.

6. The method of claim 3, wherein the moisturizer is at least one selected from the group consisting of glycerin, 1,2-hexanediol, ethylhexyl glycerin, 1,3-butylene glycol, dipropylene glycol, isopentyl glycol, propanediol, betaine, and sorbitol.

7. The method of claim 3, wherein the gellant is at least one selected from the group consisting of xanthan gum, acrylate copolymer, acrylate/C10-30 alkyl acrylate crosspolymer, sodium polyacrylate, carbomer, and cellulose gum.

8. The method of claim 1, wherein the cosmetic composition is impregnated into a mask sheet.

9. The method of claim 8, wherein the mask sheet is formed of cotton, nonwoven fabric, a rayon-containing material, or a cellulose material.
